(19) United States
(12) Patent Application Publication
(10) Pub. No.: US 2025/0241602 A1
(43) Pub. Date: Jul. 31, 2025

(54) SYSTEM AND COMPUTER PRODUCT FOR MAPPING PATIENT DATA AND REPRESENTING DATA FROM THE ELECTRONIC MEDICAL RECORD OF AN INDIVIDUAL THROUGH A PICTORIAL REPRESENTATION OF THEIR HUMAN BODY AND COLOR CODING THE SEVERITY OF THEIR AILMENTS FOR AN EASY-TO UNDERSTAND VISUAL REPRESENTATION

(71) Applicant: Kris Narayan, San Ramon, CA (US)

(72) Inventor: Kris Narayan, San Ramon, CA (US)

(21) Appl. No.: 18/165,852

(22) Filed: Feb. 7, 2023

(51) Int. Cl. A61B 5/00; G16H 10/60; G16H 50/30

(52) U.S. Cl. CPC A61B 5/744; A61B 5/743; A61B 5/7435; A61B 5/749; G16H 10/60; G16H 50/30

(57) ABSTRACT

Embodiments of the present disclosure may include a method of mapping patient data and representing data from electronic health records of an individual through a pictorial representation of their human body including receiving, over at least one communication network from each of a plurality of user computing devices operated by each of a plurality of users, electronic health records (EHR) respectively representing information from the plurality of user's electronic medical records, procured from the user's health care provider or the user's electronic health records (EHR) partners. Embodiments may also include providing, a physical representation on a display, including the user's various organs and body parts, represented in a color-coded system reporting the related electronic health records (EHR) data representing a plurality of medical records laboratory results.

### CROSS-REFERENCE TO RELATED APPLICATIONS

CLAIM OF PRIORITY. This application claims the priority of U.S. Provisional Application No. 63/372,207 filed Feb. 7, 2022, which is incorporated herein by reference.

### FIELD OF INVENTION

This invention relates to healthcare and, in particular, to an improved method and system of mapping patient data and representing a patient's condition.

### BACKGROUND OF THE INVENTION

Lifestyle factors contribute to the development of many chronic diseases, and their associated morbidity and mortality. Many of these chronic health conditions may be prevented, and an individual's span and quality of life improved through changes in lifestyle, such as consuming a healthy diet, exercising regularly, and eliminating the abuse of tobacco and alcohol.

In particular, cigarette smoking is the single most preventable cause of premature death in the United States. More than 430,000 Americans die each year from smoking-related illness, translating to one in every five deaths. An additional 110,000 people die of causes related to alcohol abuse.

Obesity is another major cause of morbidity and mortality in the United States. More than half of all adults in the United States are considered overweight or obese. Far from being a purely cosmetic disorder, obesity substantially increases morbidity and impairs the quality of life in affected individuals. It is also a risk factor for chronic diseases including hypertension, coronary heart disease, Type II diabetes, gallbladder disease, osteoarthritis and cancers of the breast, colon, and uterus. Thus, it is important to reduce body weight among obese and overweight individuals, and to prevent further weight gain in both normal and over-weight individuals.

Physical inactivity, defined as the absence of leisure time physical activity such as recreational exercise, is another major lifestyle-related risk factor for chronic health related conditions. In the United States and other developed countries, industrial automation has caused the majority of the population to be involved in sedentary occupations. Chronic conditions related to inactivity include coronary heart disease, hypertension, Type II diabetes, depression anxiety, osteoporotic hip fractures and obesity.

Lifestyle modifications alone successfully can be used to treat many chronic health conditions, without resorting to the usage of medication. For instance, mild hypertension is often controlled through lifestyle changes such as dietary modification, weight reduction, stress control, and physical activity. Weight control in itself delays and even prevents the onset of Type II Diabetes and mild hypertension.

In addition to lifestyle factors, the risk of developing many chronic health conditions is affected by a family history of disease. Diseases such as cancer, heart disease, elevated lipids, obesity, diabetes, stroke, high blood pressure, alcoholism, mental illness, and allergies all are affected lifestyle factors. With the proper management of both lifestyle modifications with family history information an overall health assessment can be made to help determine a customized overall health score.

### BRIEF SUMMARY

Embodiments of the present disclosure may include a method of mapping patient data and representing data from electronic health records of an individual through a pictorial representation of their human body including receiving, over at least one communication network from each of a plurality of user computing devices operated by each of a plurality of users, electronic health records (EHR) respectively representing information from the plurality of user's electronic medical records, procured from the user's health care provider or the user's electronic health records (EHR) partners.

Embodiments may also include providing, a physical representation on a display, including the user's various organs and body parts, represented in a color-coded system reporting the related electronic health records (EHR) data representing a plurality of medical records laboratory results. Embodiments may also include mapping, on the display of the user's whole-body avatar, the user's electronic health records (EHR) information data into a plurality of visual representations including a Severity Risk Index (SRI) representing the risk of a plurality of ailments mapped over the user's plurality of organs and body parts. Embodiments may also include providing, based on a function of the user's electronic health records (EHR) information data, the Severity Risk Index (SRI) of the plurality of medical records, and the medical records laboratory results, the user's customized overall health score.

In some embodiments, the method, creating a user's whole-body avatar by using an image-capturing device or picture file uploaded to the communication network, which may be aggregated with the electronic health records (EHR) data. In some embodiments, the method, providing further additional hidden details about the Severity Risk Index (SRI) through a clickable navigation link to data attached to the visual representation.

In some embodiments, the method, providing a history of the user's customized overall health score on the display. In some embodiments, the method, providing a plurality of score drivers. In some embodiments, the score drivers may include factors representing user-controlled parameters, activities, behaviors, and lifestyles that affect the overall health score may be derived.

In some embodiments, the electronic health records (EHR) include information representing at least one of name, DNA, birthdate, gender, age, height, weight, heart rate, blood pressure, blood type, and blood glucose. In some embodiments, the method, providing a connection to a social media network through the communication network, to present to the user at least one of the following medical statuses of the user's organ. Embodiments may also include comparison of the user's status compared to other user's status in the same age group or demographic. Embodiments may also include a list of caregivers or physicians that can help with such an ailment.

Embodiments may also include a list of services or products that can help with an ailment. Embodiments may also include a list of health care coaches that can educate the user about an ailment. Embodiments may also include a list of influencers that may be knowledgeable in such health topics. Embodiments may also include a network of other users with similar ailments. Embodiments may also include or connection to the user's family, friends or classmates from user's local health club or gym.

In some embodiments, the communication network provides a virtual assistant embedded within the user interface and connected to the communication network to interact with the user to provide voice-driven navigation of the electronic health records (EHP) using natural language processing (NLP). In some embodiments, the electronic health records (EHR) databases may be web 3.0 compliant.

Embodiments of the present disclosure may also include a system including a computing device configured to access non-transitory processor-readable media having instructions that, when executed by the computing device cause the computing device to receive, over at least one communication network from each of a plurality of user computing devices operated by each of a plurality of users, electronic health records (EHR), respectively representing information from the plurality of user's electronic medical records, procured from the user's health care provider or the user's electronic health records (EHR) partners.

Embodiments may also include provide, a physical representation on a display, including the user's various organs and body parts, represented in a color-coded system reporting the related electronic health records (EHR) data representing a plurality of medical records laboratory results. Embodiments may also include map, on the display of the user's whole-body avatar, the user's electronic health records (EHR) information data into a plurality of visual representations including a Severity Risk Index (SRI) representing the risk of a plurality of ailments mapped over the user's plurality of organs and body parts. Embodiments may also include and provide, based on a function of the user's electronic health records (EHR) data, the Severity Risk Index (SRI) of the plurality of medical records, and the plurality of medical records laboratory results, the user's customized overall health score.

In some embodiments, the user's whole-body avatar may be created by using an image capturing device or picture file uploaded to the communication network, which may be aggregated with the electronic health records (EHR) data. In some embodiments, the user's electronic health records (EHR) provide further additional hidden details about the Severity Risk Index (SRI) through a clickable navigation link to data attached to the visual representation.

In some embodiments, the processor may be configured to execute instructions that cause the processor to provide a history of the customized overall health score. In some embodiments, the processor may be configured to execute instructions that cause the processor to provide a plurality of score drivers. In some embodiments, the score drivers may include factors representing user-controlled parameters, activities, behaviors, and lifestyles that affect the overall health score may be derived.

In some embodiments, the electronic health records (EHR) include information representing at least one of name, DNA, birthdate, gender, age, height, weight, heart rate, blood pressure, blood type, and blood glucose. In some embodiments, the communication network provides a connection to a social media network through the communication network, presents to the user at least one of the following medical statuses of the user's organ. Embodiments may also include comparison of the user's status compared to other user's status in the same age group or demographic. Embodiments may also include a list of caregivers or physicians that can help with such an ailment.

Embodiments may also include a list of services or products that can help with an ailment. Embodiments may also include a list of health care coaches that can educate the user about an ailment. Embodiments may also include a list of influencers that may be knowledgeable in such health topics. Embodiments may also include a network of other users with similar ailments. Embodiments may also include or connection to the user's family, friends or classmates from user's local health club or gym.

In some embodiments, the communication network provides a connection to a social media network through the communication network, presents to the user at least one of the following medical statuses of the user's organ. Embodiments may also include comparison of the user's status compared to other user's status in the same age group or demographic. Embodiments may also include a list of caregivers or physicians that can help with such an ailment.

Embodiments may also include a list of services or products that can help with an ailment. Embodiments may also include a list of health care coaches that can educate the user about an ailment. Embodiments may also include a list of influencers that may be knowledgeable in such health topics. Embodiments may also include a network of other users with similar ailments. Embodiments may also include or connection to the user's family, friends or classmates from user's local health club or gym. In some embodiments, the communication network provides a virtual assistant embedded within the user interface to interact with the user to provide voice-driven navigation of the electronic health records (EHP) using natural language processing (NLP). In some embodiments, the electronic health records (EHR) databases may be web 3.0 compliant.

Embodiments of the present disclosure may also include a computer program product including instructions stored on a non-transitory processor-readable media and executing in a particular processor, including a decentralized distributed computer program executable in any one of a plurality of processors, including the particular processor, including a computing device configured to access non-transitory processor-readable media having instructions that, when executed by the computing device cause the computing device to receive, over at least one communication network from each of a plurality of user computing devices operated by each of a plurality of users, electronic health records (EHR) respectively representing information from the plurality of user's electronic medical records, procured from the user's health care provider or the user's electronic health records (EHR) partners.

Embodiments may also include provide, a physical representation on a display, including the user's various organs and body parts, represented in a color-coded system reporting the related electronic health records (EHR) data representing a plurality of medical records laboratory results. Embodiments may also include map, on the display of the user's whole-body avatar, the user's electronic health records (EHR) data into a plurality of visual representations including a Severity Risk Index (SRI) representing the risk of a plurality of ailments mapped over the user's plurality of organs and body parts. Embodiments may also include provide, based on a function of the user's electronic health records (EHR) data, the Severity Risk Index (SRI) of the plurality of medical records, and the medical records laboratory results, the user's customized overall health score.

In some embodiments, the user's whole-body avatar may be created by using an image capturing device or picture file uploaded to the communication network, which may be aggregated with the electronic health records (EHR) data. In some embodiments, the user's electronic health records (EHR) provide further additional details about the Severity Risk Index (SRI) through a clickable navigation link to data attached to the visual representation.

In some embodiments, the processor may be configured to execute instructions that cause the processor to provide a history of the customized overall health score. In some embodiments, the processor may be configured to execute instructions that cause the processor to provide a plurality of score drivers. In some embodiments, the score drivers may include factors representing user-controlled parameters, activities, behaviors, and lifestyles that affect the overall health score may be derived. In some embodiments, the electronic health records (EHR) include information representing at least one of name, DNA, birthdate, gender, age, height, weight, heart rate, blood pressure, blood type, and blood glucose.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a flowchart illustrating a method of mapping patient data, according to some embodiments of the present disclosure.

FIG. 2 is a block diagram illustrating a system, according to some embodiments of the present disclosure.

FIG. 3 is a block diagram further illustrating the system from FIG. 2, according to some embodiments of the present disclosure.

FIG. 4 is a block diagram further illustrating the system from FIG. 2, according to some embodiments of the present disclosure.

FIG. 5 is a block diagram illustrating a computer program product, according to some embodiments of the present disclosure.

FIG. 6 illustrates an aspect of the subject matter in accordance with one embodiment.

FIG. 7 illustrates an aspect of the subject matter in accordance with one embodiment.

FIG. 8 illustrates an aspect of the subject matter in accordance with one embodiment.

FIG. 9 illustrates an aspect of the subject matter in accordance with one embodiment.

FIG. 10 illustrates an aspect of the subject matter in accordance with one embodiment.

FIG. 11 illustrates an aspect of the subject matter in accordance with one embodiment.

FIG. 12 illustrates an aspect of the subject matter in accordance with one embodiment.

### DETAILED DESCRIPTION

In the following description, a source of content is understood to include a record of the content, such as an HTML file and a server, respectively, to provide non-limiting examples. Personalized health content is understood to refer generally to health content that is personalized to an individual's profile and general health attributes, and not merely to a health situation of a generic individual having a given health related condition. In a system for delivering health information to, and monitoring a plurality of individuals, personalized health content is understood to refer to health content customized to each individual. The statement that an input device is in communication with some data processing means is understood to mean that the data processing means is adapted to use data specified by the input either directly or indirectly. The term server is under-stood to refer to an information-generating device capable of communicating with a plurality of clients; servers include computer servers and television delivery systems. The term individual is understood to refer to a person at risk for engaging in behavior having adverse health consequences, as well as a person suffering from a chronic condition or disease.

An exemplary embodiment discussed below describes a computer-based implementation of a system and method for general personal health improvement with special emphasis on physical activity, diet, and nutrition. It will be clear to an individual with ordinary skill in the art that the present invention is suitable for preventive care directed to many other health conditions. Moreover, there are many well-known structures, interfaces and processes that are suitable for implementing the present invention.

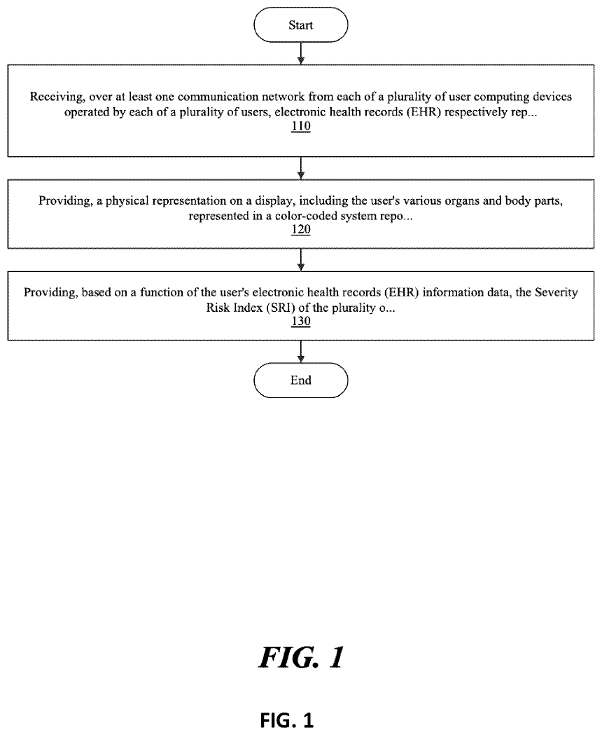

FIG. 1 is a flowchart that describes a method of mapping patient data, according to some embodiments of the present disclosure. In some embodiments, at 110, the method may include receiving, over at least one communication network from each of a plurality of user computing devices operated by each of a plurality of users, electronic health records (EHR) respectively representing information from the plurality of user's electronic medical records, procured from the user's health care provider or the user's electronic health records (EHR) partners.

In some embodiments, at 120, the method may include providing, a physical representation on a display, including the user's various organs and body parts, represented in a color-coded system reporting the related electronic health records (EHR) data representing a plurality of medical records laboratory results. At 130, the method may include providing, based on a function of the user's electronic health records (EHR) information data, the Severity Risk Index (SRI) of the plurality of medical records, and the medical records laboratory results, the user's customized overall health score. Mapping, on the display of the user's whole-body avatar, the user's electronic health records (EHR) information data into a plurality of visual representations comprising a Severity Risk Index (SRI) representing the risk of a plurality of ailments mapped over the user's plurality of organs and body parts.

In some embodiments, creating a user's whole-body avatar by using an image capturing device or picture file uploaded to the communication network, which may be aggregated with the electronic health records (EHR) data. In some embodiments, providing further additional hidden details about the Severity Risk Index (SRI) through a clickable navigation link to data attached to the visual representation.

In some embodiments, providing a history of the user's customized overall health score on the display. In some embodiments, providing a plurality of score drivers. The score drivers may comprise factors representing user-controlled parameters, activities, behaviors, and lifestyles that affect the overall health score may be derived. In some embodiments, the electronic health records (EHR) include information representing at least one of name, DNA, birthdate, gender, age, height, weight, heart rate, blood pressure, blood type, and blood glucose.

In some embodiments, providing a connection to a social media network through the communication network, to present to the user at least one of the following, the method may include performing one or more additional steps. Determining a Medical status of the user's organ; comparison of the user's status compared to other user's status in the same age group or demographic; a list of caregivers or physicians that can help with such an ailment; a list of services or products that can help with an ailment; a list of health care coaches that can educate the user about an ailment; list of influencers that may be knowledgeable in such health topics; a network of other users with similar ailments; or connection to the user's family, friends or classmates from user's local health club or gym. In some embodiments, the communication network may provide a virtual assistant embedded within the user interface and connected to the communication network to interact with the user to provide voice-driven navigation of the electronic health records (EHP) using natural language processing (NLP). In some embodiments, the electronic health records (EHR) databases may be web 3.0 compliant.

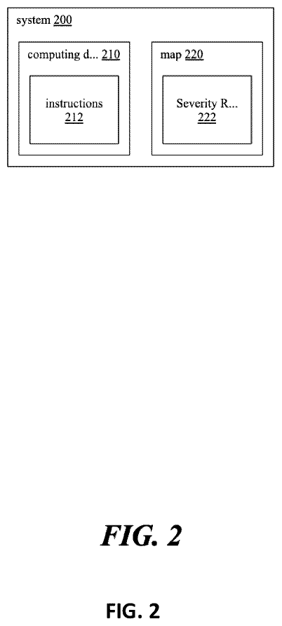

FIG. 2 is a block diagram that describes a system 200, according to some embodiments of the present disclosure. In some embodiments, the system 200 may include a computing device 210 configured to access non-transitory processor-readable media. The system 200 may also include map 220, on the display of the user's whole-body avatar, the user's electronic health records (EHR) information data into a plurality of visual representations. The computing device 210 may also include instructions 212 that, when executed by the computing device 210 cause the computing device 210 to derive a customized overall health score.

In some embodiments, Receive, over at least one communication network from each of a plurality of user computing devices operated by each of a plurality of users, electronic health records (EHR), respectively representing information from the plurality of user's electronic medical records, procured from the user's health care provider or the user's electronic health records (EHR) partners. Provide, a physical representation on a display.

In some embodiments, the user's various organs and body parts, represented in a color-coded system reporting the related electronic health records (EHR) data representing a plurality of medical records laboratory results. The map 220 may include a Severity Risk Index 222 (SRI) representing the risk of a plurality of ailments mapped over the user's plurality of organs and body parts. And provide, based on a function of the user's electronic health records (EHR) data, the Severity Risk Index 222 (SRI) of the plurality of medical records, and the plurality of medical records laboratory results, the user's customized overall health score.

In some embodiments, the user's whole-body avatar may be created by using an image capturing device or picture file uploaded to the communication network, which may be aggregated with the electronic health records (EHR) data. In some embodiments, the user's electronic health records (EHR) provide further additional hidden details about the Severity Risk Index 222 (SRI) through a clickable navigation link to data attached to the visual representation.

In some embodiments, the processor may be configured to execute instructions that cause the processor to provide a history of the customized overall health score. In some embodiments, the processor may be configured to execute instructions that cause the processor to provide a plurality of score drivers. The score drivers may also include factors representing user-controlled parameters, activities, behaviors, and lifestyles that affect the overall health score may be derived.

In some embodiments, the electronic health records (EHR). In some embodiments, the communication network may provide a virtual assistant embedded within the user interface to interact with the user to provide voice-driven navigation of the electronic health records (EHP) using natural language processing (NLP). In some embodiments, the electronic health records (EHR) databases may be web 3.0 compliant.

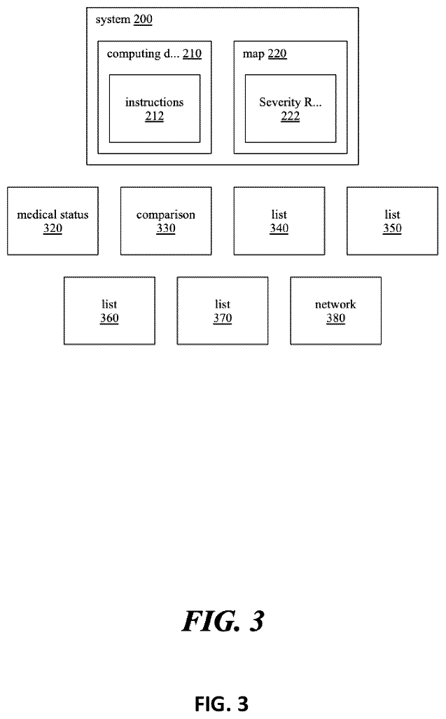

FIG. 3 is a block diagram that further describes the system 200 from FIG. 2, according to some embodiments of the present disclosure. In some embodiments, the communication network may provide a connection to a social media network through the communication network, presents to the user at least one of the following: connection to the user's family, friends or classmates from user's local health club or gym.

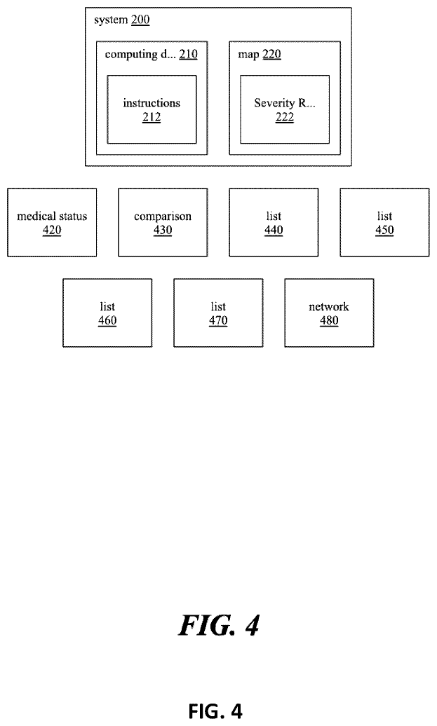

FIG. 4 is a block diagram that further describes the system 200 from FIG. 2, according to some embodiments of the present disclosure. In some embodiments, the communication network may provide a connection to a social media network through the communication network, presents to the user at least one of the following: connection to the user's family, friends or classmates from user's local health club or gym.

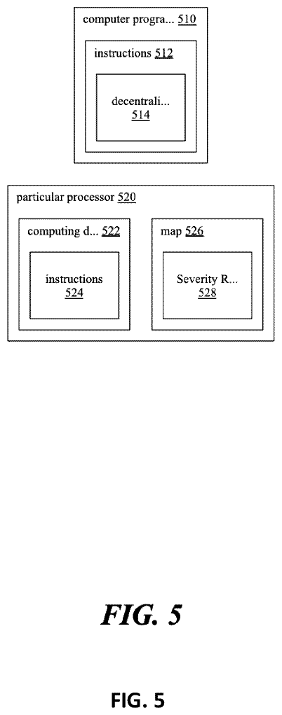

FIG. 5 is a block diagram that further describes the system 200 from FIG. 2, according to some embodiments of the present disclosure. In some embodiments, the communication network may provide a connection to a social media network through the communication network, presents to the user at least one of the following: connection to the user's family, friends or classmates from user's local health club or gym.

FIG. 5 is a block diagram that further describes the system 200 from FIG. 2, according to some embodiments of the present disclosure. In some embodiments, the communication network may provide a connection to a decentralized system for aggregating user patient information on a blockchain. The blockchain and web 3.0 modules will allow for the patient to be in control of their medical data to ensure they are using the best information and resources to facilitate an accurate customized overall health score.

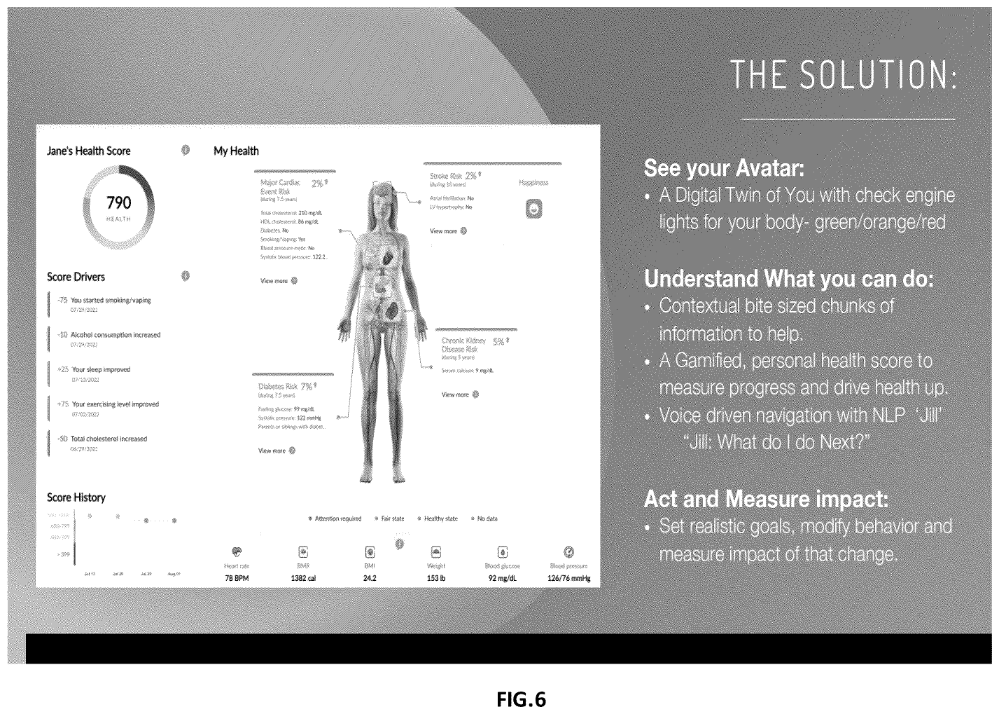

FIG. 6 is a view of the dashboard that displays an avatar of the patient mobile application dashboard that displays the patient's electronic health records (EHR) data aggregated to advise the patient of severity of any ailments, specific organ and body parts status and the patient's customized overall health score.

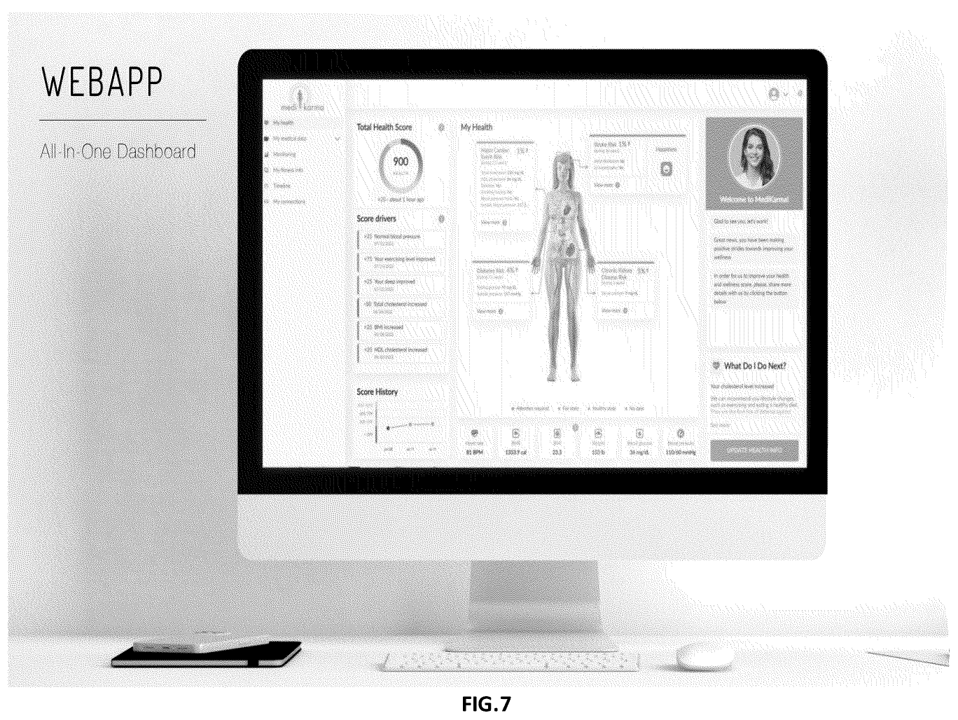

FIG. 7 is a view of the all-in-one dashboard on a computer display. FIG. 7 also depicts a rendering of “Jill” the virtual assistant that assists with advising the patient of next steps and answers any questions about the electronic health records data displaying the severity risk indices. Each of the severity risk indices are specific to different organs and body parts of the patient.

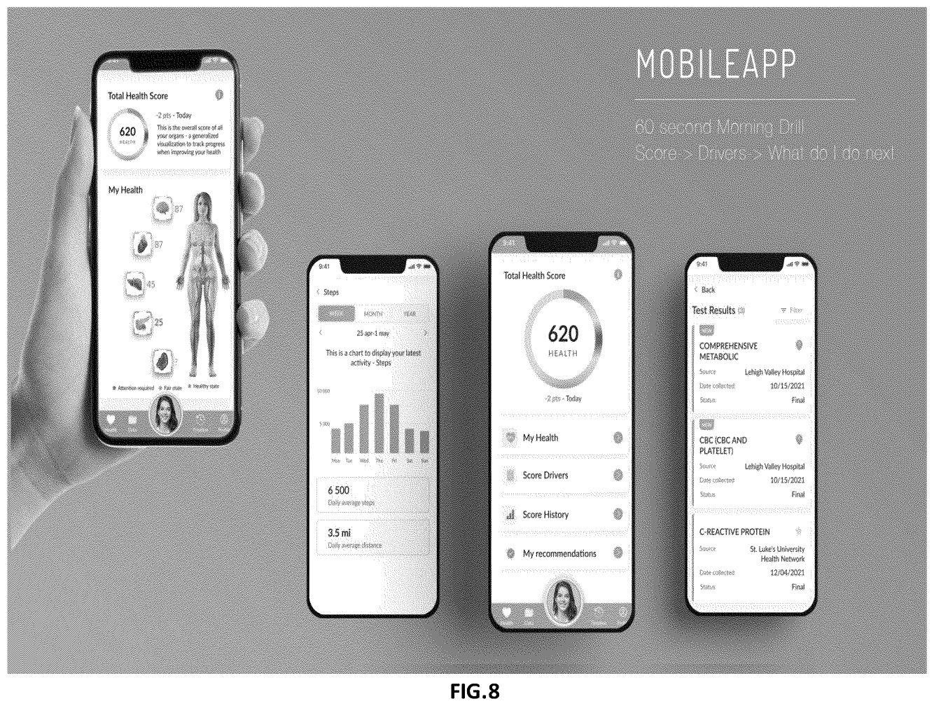

FIG. 8 is a view of the mobile application with various views of electronic health records data being displayed on a mobile device as the application would be used by the patient.

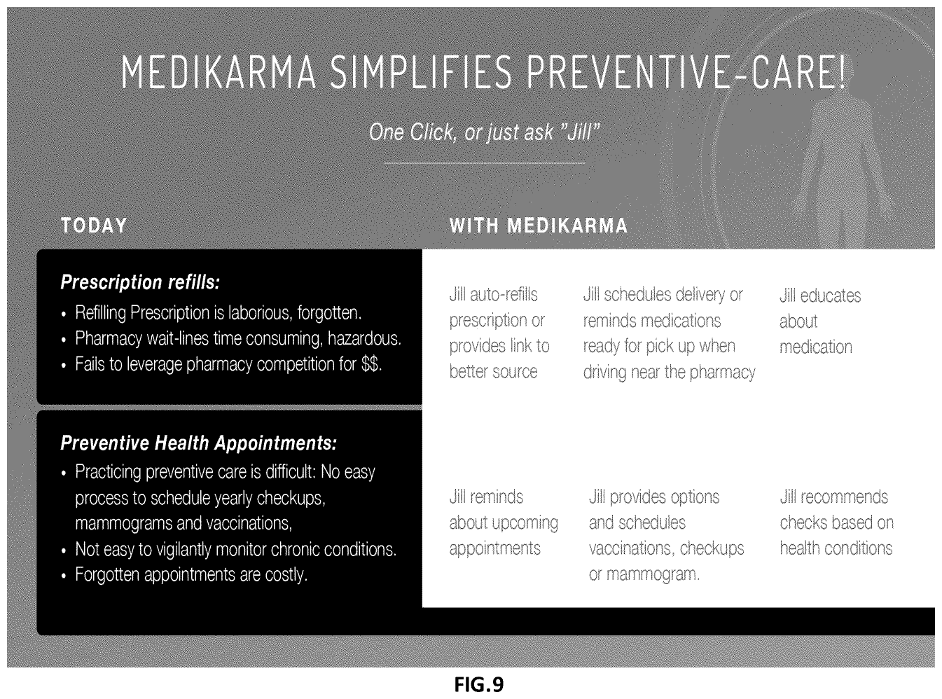

FIG. 9 is a view of the steps of a virtual assistant algorithm providing advice to the patient on the prescription refills and provides a link to better sources, if they exist. The virtual assistant educates the patient about the medication and reminds the patient of upcoming appointments, vaccinations, checkups, or mammogram. Virtual Assistant “Jill” will make recommendations that would be in the best interest of the patient.

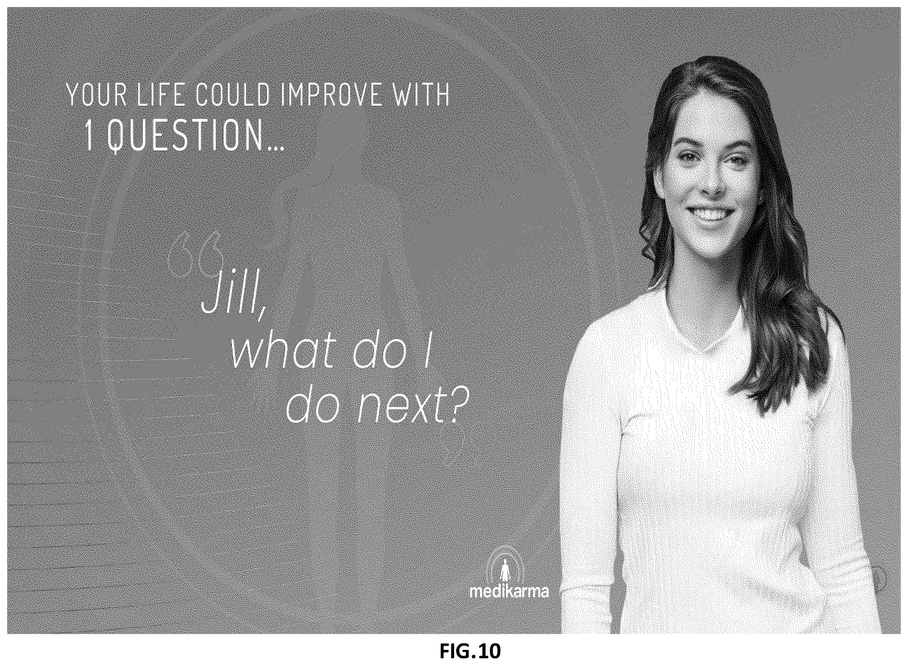

FIG. 10 is a view a hypothetical avatar of the Virtual Assistant “Jill”. Jill, like other Virtual Assistants that use Natural Language Processing, interacts with the patient to ensure a simple response to help ensure that the patient is able to understand the

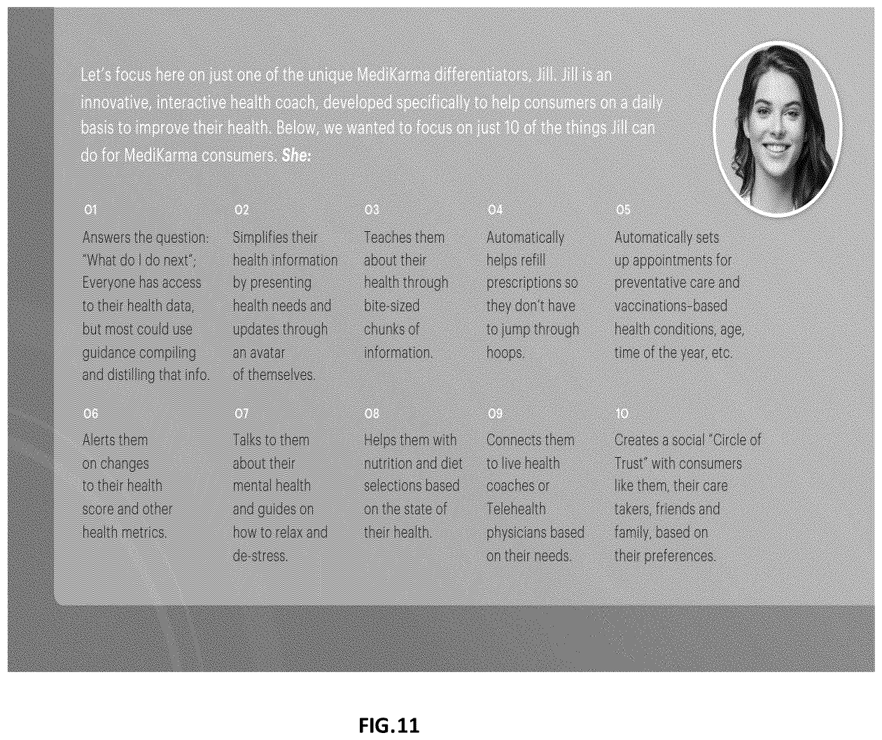

FIG. 11 is a view of the various tasks that the Virtual Assistant “Jill” can perform for the patient to facilitate the translation of the electronic medical data (EHR) and the customized overall health score.

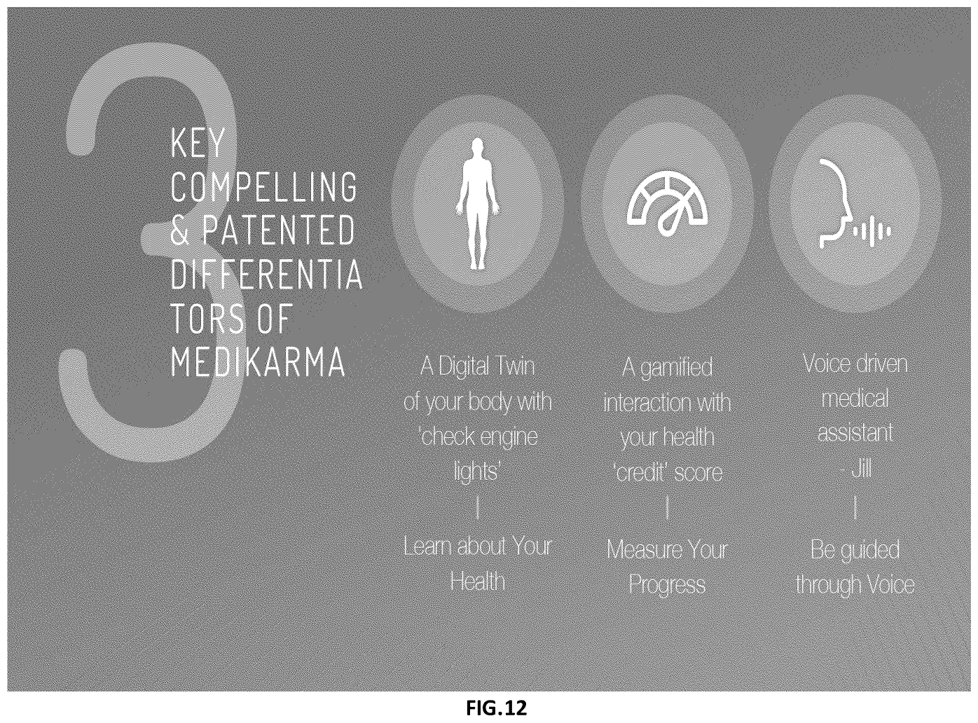

FIG. 12 is a description of the three differentiators from the claimed invention. Medical records are analyzed and aggregated to provide a digital twin of a patient's body with “check engine lights” to educate the patient about their health. A social media platform is connected to the application to create a gamified interaction with the patient's customized overall health score to know the status of any ailments the patient may be suffering to help the patient measure their progress. The patient interacts with the virtual assistant “Jill,” and it provides guidance on the patient's health progress.

It will be clear to one skilled in the art that the above invention may be altered in many ways without departing from the scope of the invention. Various relative arrangements of inputs and processing means are possible for generating personalized health content. In addition to general health improvement, various diseases and behaviors are amenable to preventive care according to a method of the present invention, including asthma, hypertension, cardio-vascular disease, eating disorders, breathing disorders, blood pressure disorders, heart conditions, HIV, mental health dis-orders, smoking, and drug or alcohol abuse.

While the preferred embodiment of the invention has been illustrated and described, as noted above, many changes can be made without departing from the spirit and scope of the invention. Accordingly, the scope of the invention is not limited by the disclosure of the preferred embodiment.

What is claimed is:

1. A method of mapping patient data and representing data from electronic health records of an individual through a pictorial representation of their human body comprising:
receiving, over at least one communication network from each of a plurality of user computing devices operated by each of a plurality of users, electronic health records (EHR) respectively representing information from the plurality of user's electronic medical records, procured from the user's health care provider or the user's electronic health records (EHR) partners; providing, a physical representation on a display, including the user's various organs and
body parts, represented in a color-coded system reporting the related electronic health records (EHR) data representing a plurality of medical records laboratory results; mapping, on the
display of the user's whole-body avatar, the user's electronic health records (EHR) information data into a plurality of visual representations comprising a Severity Risk Index (SRI) representing the risk of a plurality of ailments mapped over the user's plurality of organs and body parts; and providing, based on a function of the user's electronic health records (EHR) information data, the Severity Risk Index (SRI) of the plurality of medical records, and the medical records laboratory results, the user's customized overall health score.

2. The method of claim 1, creating a user's whole-body avatar by using an image-capturing device or picture file uploaded to the communication network, which is aggregated with the electronic health records (EHR) data.

3. The method of claim 1, providing further additional hidden details about the Severity Risk Index (SRI) through a clickable navigation link to data attached to the visual representation.

4. The method of claim 1, providing a history of the user's customized overall health score on the display.

5. The method of claim 1, providing a plurality of score drivers, wherein the score drivers comprise factors representing user-controlled parameters, activities, behaviors, and lifestyles that affect the overall health score is derived.

6. The method of claim 1, wherein the electronic health records (EHR) include information representing at least one of name, DNA, birthdate, gender, age, height, weight, heart rate, blood pressure, blood type, and blood glucose.

7. The method of claim 1, providing a connection to a social media network through the communication network, to present to the user at least one of the following: medical status of the user's organ; comparison of the user's status compared to other user's status in the same age group or demographic; a list of caregivers or physicians that can help with such an ailment; a list of services or products that can help with an ailment; a list of health care coaches that can educate the user about an ailment; a list of influencers that are knowledgeable in such health topics; a network of other users with similar ailments; or connection to the user's family, friends or classmates from user's local health club or gym.

8. The method of claim 1, wherein the communication network provides a virtual assistant embedded within the user interface and connected to the communication network to interact with the user to provide voice-driven navigation of the electronic health records (EHP) using natural language processing (NLP).

9. The method of claim 1, wherein the electronic health records (EHR) databases are web 3.0 compliant.

10. A system comprising:
a computing device configured to access non-transitory processor-readable media having instructions that, when executed by the computing device cause the computing device to:
receive, over at least one communication network from each of a plurality of user computing devices operated by each of a plurality of users, electronic health records (EHR), respectively representing information from the plurality of user's electronic medical records, procured from the user's health care provider or the user's electronic health records (EHR) partners; provide, a physical representation on a display, including the user's various organs and
body parts, represented in a color-coded system reporting the related electronic health records (EHR) data representing a plurality of medical records laboratory results;
map, on the display of the user's whole-body avatar, the user's electronic health records (EHR) information data into a plurality of visual representations comprising a Severity Risk Index (SRI) representing the risk of a plurality of ailments mapped over the user's plurality of organs and body parts; and provide, based on a function of the user's electronic health records (EHR) data, the Severity Risk Index (SRI) of the plurality of medical records, and the plurality of medical records laboratory results, the user's customized overall health score.

11. The system of claim 10, wherein the user's whole-body avatar is created by using an image-capturing device or picture file uploaded to the communication network, which is aggregated with the electronic health records (EHR) data.

12. The system of claim 10, wherein the user's electronic health records (EHR) provide further additional hidden details about the Severity Risk Index (SRI) through a clickable navigation link to data attached to the visual representation.

13. The system of claim 10, wherein the processor is configured to execute instructions that cause the processor to provide a history of the customized overall health score.

14. The system of claim 10, wherein the processor is configured to execute instructions that cause the processor to provide a plurality of score drivers, wherein the score drivers comprise factors representing user-controlled parameters, activities, behaviors, and lifestyles that affect the overall health score is derived.

15. The system of claim 10, wherein the electronic health records (EHR) include information representing at least one of name, DNA, birthdate, gender, age, height, weight, heart rate, blood pressure, blood type, and blood glucose.

16. The system of claim 10, wherein the communication network provides a connection to a social media network through the communication network, presents to the user at least one of the following: medical status of the user's organ; comparison of the user's status compared to other user's status in the same age group or demographic; a list of caregivers or physicians that can help with such an ailment; a list of services or products that can help with an ailment; a list of health care coaches that can educate the user about an ailment; a list of influencers that are knowledgeable in such health topics; a network of other users with similar ailments; or connection to the user's family, friends or classmates from user's local health club or gym.

17. A computer program product comprising instructions stored on a non-transitory processor readable media and executing in a particular processor, comprising a decentralized distributed computer program executable in any one of a plurality of processors, including the particular processor, including:
a computing device configured to access non-transitory processor-readable media having instructions that, when executed by the computing device cause the computing device to:
receive, over at least one communication network from each of a plurality of user computing devices operated by each of a plurality of users, electronic health records (EHR) respectively representing information from the plurality of user's electronic medical records, procured from the user's health care provider or the user's electronic health records (EHR) partners; provide, a physical representation on a display, including the user's various organs and
body parts, represented in a color-coded system reporting the related electronic health records (EHR) data representing a plurality of medical records laboratory results; map, on the display of the user's whole-body avatar, the user's electronic health records (EHR) data into a plurality of visual representations comprising a Severity Risk Index (SRI) representing the risk of a plurality of ailments mapped over the user's plurality of organs and body parts; and provide, based on a function of the user's electronic health records (EHR) data, the Severity Risk Index (SRI) of the plurality of medical records, and the medical records laboratory results, the user's customized overall health score.

18. The computer program product of claim 17, wherein the user's whole-body avatar is created by using an image-capturing device or picture file uploaded to the communication network, which is aggregated with the electronic health records (EHR) data.

19. The computer program product of claim 17, wherein the user's electronic health records (EHR) provide further additional details about the Severity Risk Index (SRI) through a clickable navigation link to data attached to the visual representation.

20. The computer program product of claim 17, wherein the processor is configured to execute instructions that cause the processor to provide a history of the customized overall health score.

21. The computer program product of claim 17, wherein the processor is configured to execute instructions that cause the processor to provide a plurality of score drivers, wherein the score drivers comprise factors representing user-controlled parameters, activities, behaviors, and lifestyles that affect the overall health score is derived.

22. The computer program product of claim 17, wherein the electronic health records (EHR) include information representing at least one of: name, DNA, birthdate, gender, age, height, weight, heart rate, blood pressure, blood type, and blood glucose.

23. The computer program product of claim 17, wherein the communication network provides a connection to a social media network through the communication network, presents to the user at least one of the following: medical status of the user's organ; comparison of the user's status compared to other user's status in the same age group or demographic; a list of caregivers or physicians that can help with such an ailment; a list of services or products that can help with an ailment; a list of health care coaches that can educate the user about an ailment; a list of influencers that are knowledgeable in such health topics; a network of other users with similar ailments; or connection to the user's family, friends or classmates from user's local health club or gym.

24. The computer program product of claim 17, wherein the communication network provides a virtual assistant embedded within the user interface to interact with the user to provide voice-driven navigation of the electronic health records (EHP) using natural language processing (NLP).

25. The computer program product of claim 17, wherein the electronic health records (EHR) databases are web 3.0 compliant.
